(19) United States
(12) Patent Application Publication
(10) Pub. No.: US 2010/0280404 A1
(43) Pub. Date: Nov. 4, 2010

(54) ELECTRICAL IMPEDANCE TOMOGRAPHY EQUIPMENT

(75) Inventors: Edward Lazo Maktura, Sao Paulo (BR); Albert Holzhacker, Sao Paulo (BR)

Correspondence Address: BIRCH STEWART KOLASCH & BIRCH, PO BOX 747, FALLS CHURCH, VA, 22040-0747, US

(73) Assignee: Dixtal Biomedica Industria E Comercio Ltda., San Paulo (BR)

(21) Appl. No.: 12/681,361

(22) PCT Filed: Sep. 25, 2008
(86) PCT No.: PCT/BR08/00289

§ 371 Date: Jun. 22, 2010

(30) Foreign Application Priority Data
Oct. 3, 2007 (BR) PI0704023-7

(51) Int. Cl. A61B 5/053

(52) U.S. Cl. 600/547

(57) ABSTRACT

There is described an electrical impedance tomography equipment, intended for providing images representing the inside of a portion of the body of a patient by means of electrical impedance measurements taken between electrodes positioned on the skin, comprising a plurality of acquisition units (15, 27) provided with circuits for processing low level analog signals picked up by the cables (13, 29) that interconnect the said equipment with the electrodes (14) applied to the patient, the said acquisition units being substantially symmetrically arranged to provide as a result a substantially equal irradiation of the heat generated in these units, easing the calibration of the analog channels and common mode rejection. The equipment further comprises at least one plane constituted by a printed circuit board whereto the said units are electrically connected, the said plane comprising complementary elements and devices, such as the electrical connection terminals for the said cables.

### FIELD OF THE INVENTION

The present invention refers to the provision of images representative of the inside of a part of the body of a patient, and particularly refers to such imaging by means of measurements of electrical impedance between electrodes positioned on the skin.

### DESCRIPTION OF THE PRIOR ART

The term “tomography” is used to designate methods that provide images of the inside of the body on a predetermined plane. The most widely known methods for the provision of such images make use of X-rays, magnetic resonance, positron emission, ultrasound, and electrical impedance measurements. The latter provides the advantage that, in addition to being non-invasive, it does not generate any type of potentially harmful stimuli, such as occurs with X-rays or strong magnetic fields. Furthermore, as compared with these latter, it uses equipment of substantially lower cost, and can be used for uninterrupted monitoring of the patient's conditions, which is not feasible with the cited equipment.

The principles of electrical impedance tomography were established at the end of the 1970's and beginning of the 1980's, and as one of the pioneering patents in that field there may be cited document U.S. Pat. No. 4,617,939, published in 1986 with the title “Tomography”. According to what is described in the cited document, the method used consists in positioning a plurality of electrodes at regular intervals in contact with the skin of the patient, inducing an electric current of 50 kHz and 4 mA in the body by applying an electrical voltage of 3V between adjacent pairs of electrodes and measuring the voltages between the remaining pairs of adjacent electrodes. In the embodiment as described, there are used 16 electrodes, resulting in a total 1456 voltage values. The results of these measurements are analyzed by means of an algorithm that provides a representation, in two dimensions, of the impedances occurring in the plane of the electrodes.

Documents U.S. Pat. No. 4,920,490 and U.S. Pat. No. 5,381,333 describe the simultaneous application of current to a plurality of electrodes, where the voltages of the electrodes are measured with regard to a single grounded electrode that is used as a reference.

Document U.S. Pat. No. 4,272,624 describes the simultaneous application of current to a plurality of electrodes, where the voltages of the electrodes are measured against a single grounded electrode used as a reference.

The paper presented by P. Hua, J. G. Webster, W. J. Tompkins at the Ninth Annual Conference of the IEEE Engineering in Medicine and Biology Science in 1987, with the title “Effect of the measurement method on noise handling and image quality of EIT imaging” describes the application of a current using a pair of electrodes, with successive measurement of the resulting voltages for all the remaining pairs of adjacent electrodes, with a mention to the effect that the current may be applied between two diametrically opposite electrodes.

The paper published in IEEE Transactions on Biomedical Engineering, vol. 39, No. 7 with the title “Measuring Lung Resistivity Using Electrical Impedance Tomography”, which authors are E. J. Woo et al., describes the application of current using an optimum pattern, measuring the resulting limit voltages against a common reference.

The article “Finite Element Modeling of Electrode-Skin Contact Impedance in Electrical Impedance Tomography”, by P. Hua et al., published in IEEE Transactions on Biomedical Engineering, vol. 40, No. 4, describes a process whereby there is applied current at neighboring electrodes, measuring the resulting voltages with regard to a common reference electrode.

The paper “Image reconstruction using non-adjacent drive configurations” by N. J. Avis and D. C. Barber, published in the periodical Physiological Measurement, vol. 15, 1994, describes a method whereby the currents between electrodes are applied in adjacent, crosswise and polar configurations, with measurement of the voltages between pairs of adjacent electrodes that carry no current.

In Brazilian patent application No. PI 0306103-5, with the title “Equipamento para realizar tomografia de impedância elétrica” [Equipment for Performance of Electrical Impedance Tomography], published in Aug. 16, 2005, there is described a sophisticated system that, according to the authors, is intended to provide up to 200 images per second. To that end, there is used a dedicated voltage measuring unit for each input channel, each unit comprising an A/D converter with at least 16-bit resolution, capable of performing 5 million measurements per second and transmitting the data using a communication network at a rate of 80 Mb/s, where the voltage measurements in the electrodes are performed simultaneously at each stimulus. The stimulus may consist in a wave of arbitrary shape, generated by a processor and processed by a D/A converter, followed by a programmable attenuator and complementary amplifiers to generate bipolar current signals. The document provides the use of signals in the range from 10 kHz to 2.5 MHz. A measurement means coupled at the output of the amplifiers provides a signal that is fed back through the processor, and adjusts the attenuator to provide the intended current at the output. The bipolar excitation signals are fed to two multiplexers that feed the currents, in programmed fashion, to the terminals applied to the patient, such programming allowing the application of the excitation currents to adjacent or opposed terminals, successively displacing the position thereof during performance of the tomography.

In addition to not providing a sufficiently detailed description of the method used, the above document describes the system only at the block level and does not comprise any information relative to details of circuits or physical distribution of the units. Furthermore, no consideration was given to the fact that this type of equipment is not used in isolated environments, but rather in an electromagnetically polluted environment, due to the simultaneous use of equipment such as cardiac monitors, ventilators, blood pressure and oxygenation measuring devices, etc. In the said document no account was taken of the fact that high frequencies interfere with and are subject to interference from other circuits in radiated fashion or coupled fashion by parasitic capacitances and eddy inductances that shift the phase and gain of the system and generate cross modulation, overvoltage, transients and others, corrupting both analog and digital signals. Such interferences are observed in the form of noise that affects the reconstruction of the image, impairing or even rendering unfeasible the analysis of certain physiological variations. In this regard, the presently cited application does not provide any information relative to the measures that might be taken toward reducing the effect of such interferences.

In application No. PI 0306103-5 there is further mentioned that the image resolution quality is enhanced in relation to that which is obtained using known techniques, since the system that constitutes the object of the application can be configured to use up to 64 electrodes. However, the increase in the number of electrodes is faced with a practical limitation, that is the decrease of signal/noise ratio arising from such increase. Furthermore, the resolution is limited by the three-dimensional scattering of the current, and therefore the improvement in resolution cannot be indefinitely increased by simply increasing the number of electrodes.

### OBJECTS OF THE INVENTION

In view of what has been set forth above, a first object of the invention consists in the provision of an electrical impedance tomography system wherein the resolution may be improved without significant impact on the signal/noise ratio.

One other object of the invention consists in the provision of a system that is less susceptible to interference, either from radiation from other equipment, or from coupling and/or imbalance caused by eddy current inductance and parasitic capacitances.

### BRIEF DESCRIPTION OF THE INVENTION

The objects cited herein and others are achieved by the invention by means of the use of hardware structures that provide maximum symmetry between the functional blocks, both analog and digital. Thus, the proposed equipment comprises a plurality of low level signal acquisition units, along with complementary elements and devices, with the said units provided in substantially symmetric arrangement in relation to at least one axis of symmetry which can be vertical or horizontal.

According to another characteristic of the invention, the said complementary elements and devices are provided in at least one plane comprised by a printed circuit board, whereto are electrically connected, in symmetric fashion, the said acquisition units.

Advantageously, the substantially symmetric distribution brings as a consequence a substantially equal irradiation of the heat generated in these units, easing the task of calibration of the analog signals and of common mode rejection.

According to another characteristic of the invention, the equipment comprises two planes containing the said elements and devices, the first substantially intended for processing the analog signals and the second substantially intended for processing the digital signals.

According to another characteristic of the invention, the distance between the said first and second planes corresponds to one of the physical dimensions of the said acquisition units, such as the height thereof, allowing to connect the same electrically both to the first and to the second planes.

According to another characteristic of the invention, each acquisition unit comprises one or more printed circuit boards.

According to another characteristic of the invention, the said first and second planes are oriented horizontally.

According to another characteristic of the invention, the said first and second planes are oriented vertically.

According to another characteristic of the invention, the said first and second planes have identical dimensions and shapes.

According to another characteristic of the invention, the said first and second planes have different dimensions or shapes.

According to another characteristic of the invention, the rejection of common mode interferences is eased by the fact that all the lines that carry low level signals have the same physical length, such fact applying both to the cables that connect the equipment to the patient and to the internal conductors of the equipment.

### DESCRIPTION OF THE FIGURES

The various advantages and characteristics of the inventions will become more apparent from the description of a preferred embodiment given as example and not intended to be construed in any limitative sense, and of the figures that refer thereto, wherein:

FIG. 1 is a schematic illustration of the principle of symmetric geometry distribution proposed by the invention, as well as the path traveled by the signals acquired by the system.

FIG. 2 is an illustration, also in a schematic and simplified manner, of the path traveled by the excitation signals to be injected into the patient.

FIG. 3 is a simplified exploded view illustrating the spatial arrangement of the functional units that make up the equipment in a preferred embodiment thereof.

FIG. 4 is a perspective view illustrating the equipment that constitutes the object of the present invention.

FIG. 5 shows an exemplary drawing of the conductive strips on the lower face of the equipment.

### DETAILED DESCRIPTION OF THE INVENTION

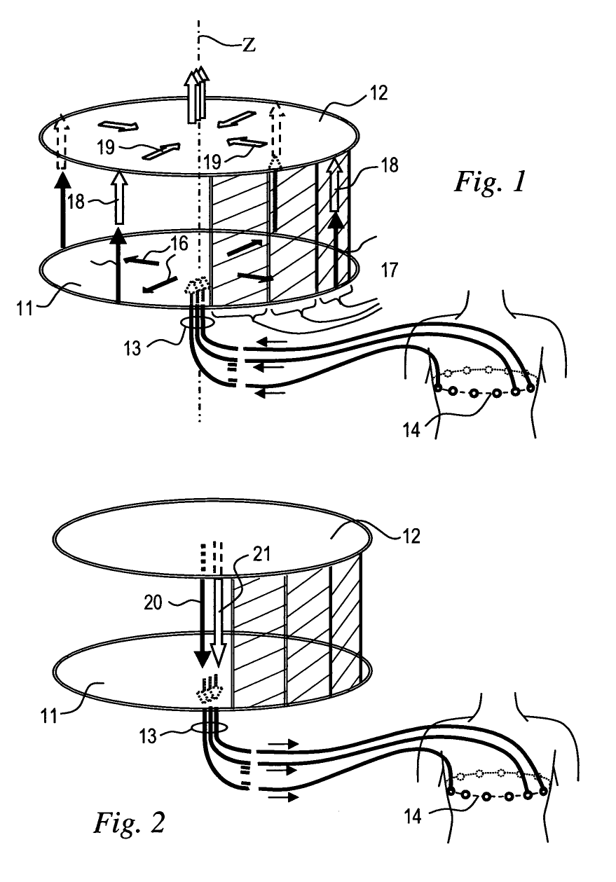

FIGS. 1 and 2 are schematic illustrations of the principles of spatial distribution of the functional blocks that represent the equipment. These blocks comprise a first plane 11 and a second plane 12, the first processing the analog signals and the second processing the digital signals. In a preferred embodiment, the first plane 11 is oriented horizontally and is provided in the lower position, while the second plane 12 is provided in the upper position. However, the principles of the invention apply as well to planes with different orientations, and also to planes with different shapes and/or dimensions.

In FIGS. 1 and 2 the said lower plane is provided with means for connection to the bundle of cables 13 whereby is provided the connection of the system to the set of electrodes 14 that are applied to the patient. Preferably the said connection means are located at the center of the said lower plane 11, and the signals picked up by the electrodes and brought over the cables are distributed in radial symmetry to the periphery of the said lower plane, whereon are positioned the signal acquisition units 15, whereof only three are represented symbolically in the figure, for a question of clarity. In these units the signals are initially amplified by analog amplifiers, and are subsequently digitized, as indicated by the arrows. In order to provide a better understanding of the process, the arrows depicted with a full line 16, 17 represent symbolically the path traveled by the analog signals, while the blank arrows 18, 19 illustrate the path traveled by the digital signals. These latter are transferred from the units 15 to the periphery of the upper plane 12 and therefrom to the central part of that plane, wherefrom they are sent to the computing and image display means (not shown).

FIG. 2 is a schematic representation of the generation of the excitation signals, that are digitally synthesized by means of circuits located in the upper plane 12. In a preferred embodiment, this signal may be a sine wave which upon being synthesized and filtered, is transferred to the upper plane, as indicated by arrow 20. Together with this signal, the upper plane sends the digital commands of the injection sequence, arrow 21, to the said lower plane wherein are located the multiplexers as well as the current sources that transform the signal into voltage produced in the upper plane in the currents that will be applied to the patient, by means of the set of cables 13 and electrodes 14.

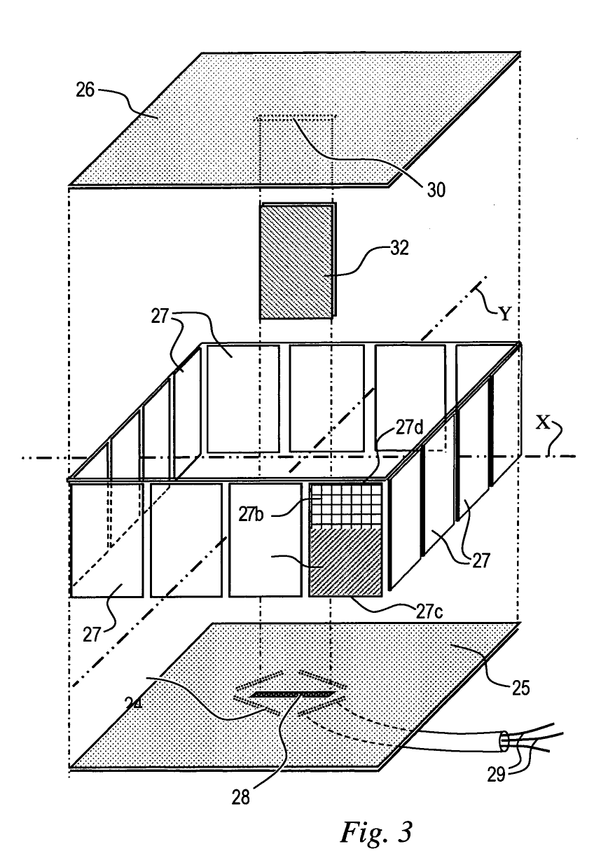

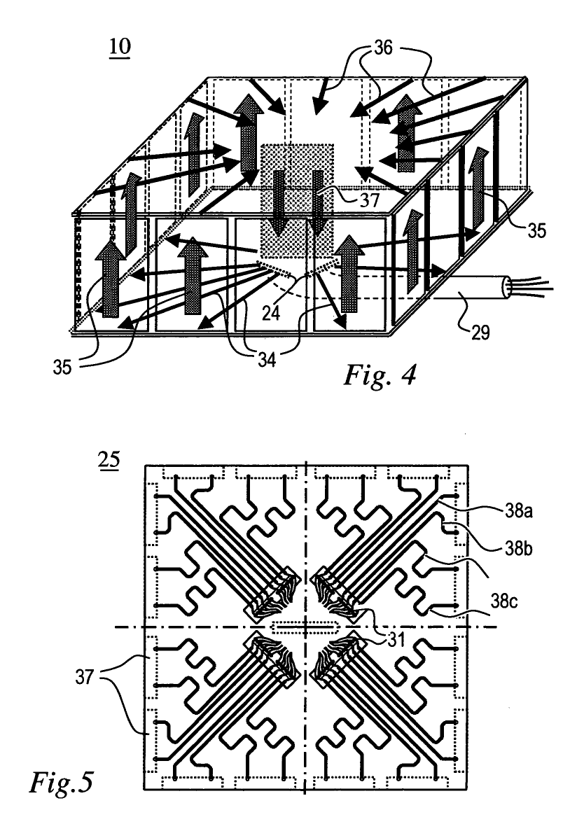

The geometry illustrated in FIGS. 1 and 2 is substantially cylindrical, or approximately prismatic, with the processing units 15 arranged equidistantly in relation to the vertical axis of symmetry Z. However, this form may be difficult to provide in industrial scale; and there may adopted other forms such as regular polygonal shapes (hexagonal, octagonal, etc.) without loss of symmetry. In a preferred embodiment, these units are arranged uniformly along the sides of a square shape, as shown in FIGS. 3 and 4. As illustrated, this arrangement maintains the symmetry of the processing units 27 with relation to the axes X and Y, as well as the symmetry of the lower plane 25 and the upper plane 26 with relation to the plane formed by the said axes. The peripheral position of the processing units 27, along the borders of the squares formed by the lower and upper planes, is advantageous from the thermal perspective, providing similar heat irradiation conditions, whereby all these units operate at substantially the same temperature.

FIG. 3 is an exploded view of the equipment in its preferred embodiment, showing the same to comprise four types of boards, to with:
a plurality of acquisition units 27 for processing low level signals, each of such units comprising two acquisition blocks, each of these latter being associated with an excitation/pickup cable;
a first plane 25, comprised of a square-shaped board, comprising the multiplexing circuits and current source. This board is provided with connectors along its borders, whereto are coupled the said units 27. In the exemplary embodiment shown, the said first plane is in a lower position and the board that constitutes the said plane 25 is provided at its central region with sets of terminals 24 for connection to the proximal ends of the signal injection or pickup cables;
a second plane 26, provided parallel to the said first plane, comprised of a square-shaped board provided along the borders thereof with means of contact with the upper ends of the units 27. In the exemplary embodiment shown, this second plane, which comprises the digital signal processing circuits, is provided in an upper position;
an interface board 32 that electrically connects the centers of the boards that constitute the upper plane 26 and the lower plane 25.

As shown in this figure as well as in FIG. 4, the said boards are mounted in a “cubic” volume arrangement, or rather in a square-shaped parallelepiped form. As cited hereinabove, the lower (external) face of the lower board 25 is provided with four connection bars 24 to which terminals are electrically connected the proximal ends of the interconnection cables, collectively represented as a bundle of cables 29 that carry the excitation signals as well as the responses picked up by the electrodes provided in contact with the skin of the patient, there being used thirty-two cables in the exemplary embodiment of the invention.

In the present exemplary embodiment, the units 27 constitute modules formed by two acquisition blocks, and therefore two cables are associated with each module. Other embodiments are possible, and each module can be associated to 1, 4 or more cables, corresponding to the number of acquisition blocks per module. As indicated in the figure by means of a broken line, the cable bundle 29 is brought beneath the lower board 25 and the cables are individually connected to the corresponding terminals in the bars 24.

According to the illustration of FIG. 4, the signals carried by the said cables (not depicted in this figure) are conveyed from the terminal bars 24 at the center of the plane 25 to the borders of that plane, such path being symbolically represented by the arrows 34. As indicated by FIG. 5, at the perimeter of the board 25 there are provided connectors 37 whereinto are inserted the lowers borders of the units 27. These units comprise at the lower region thereof the high sensibility analog circuits for amplification of the signals picked up by the electrodes (not illustrated). Such circuits comprise means for phase and gain adjustment that allow to eliminate common mode interferences, which means can be provided by manually adjustable resistors (“trimpots”) or digital devices that perform the same function, where in these latter the adjustments are stored in memory. The upper portions 27b of the said acquisition units comprise the A/D converter circuits that digitally encode the signals amplified by the analog portion 27a, subsequently conveying the same to the upper plane 26, as symbolically represented by the arrows 35.

In order to preserve the electrical symmetry between the conductors that carry low level signals, the layouts of the strips that join the terminal bars 31 to the connectors 37 are configured to compensate the differences of geometrical distances between the said terminals and the connectors located at different positions along the border of the board 25. Thus, according to the illustration of FIG. 5, the layouts of the strips 38b, 38c and 38d are configured to result in the same length as strip 38a. With this layout, the parasitic inductances and capacitances, as well as the noise induced by external electromagnetic fields become approximately identical, thereby allowing the reduction of noise caused by common mode rejection.

Thus, from the electrical point of view, all the acquisition blocks are equidistant from the center of the boards 25 and 26, and therefore also from the interface board 28 that interconnects the upper and lower boards. Furthermore, the spatial arrangement of the said blocks provides a uniform distribution of irradiated and conducted heat, such that all analog circuits operate at substantially the same temperature, reducing the variation of common mode rejection caused by temperature.

The part of the acquisition unit 27 located nearest to the board 25, which in the illustrated embodiment is the lower region 27a, contains the circuits for analog processing of the input signal, comprising the means for individual compensation of phase and gain in order to eliminate common mode interference.

The upper portion 27b of the boards of the said acquisition units contains the circuits for digital processing of the signal, comprising the conversion thereof from analog to digital, with subsequent transfer of this digitized signal to the demodulation and control plane by way of connectors (not shown) located at the borders of the plate 26. This plate comprises registries that store the values of the signals output from the pickup electrodes, which reading is performed simultaneously by the acquisition units. The board 26 further comprises the excitation signal generator circuit and the readout sequencer. The arrows 20 and 21 in FIGS. 2 and 37 in FIG. 4 are symbolic representations of the path respectively traveled by the excitation signal and the information on the readout sequence.

Although the invention has been described based on a preferred embodiment thereof, it should be pointed out that there may be introduced modifications in its basic concept, without the inventive concept falling outside of the scope of the invention.

Thus, for example, the upper and lower planes that make up the set may have different dimensions, thereby configuring, for example, a solid with a frustopyramidal shape. In alternative embodiments, the acquisition units may be provided symmetrically, however in radial orientation with relation to the center of the planes. Furthermore, in another embodiment, there may be employed only one of the planes, such as the lower plane, and in such case the digital processing circuits in the acquisition units would be located beside the analog processing circuits instead of above the same as adopted in the exemplary embodiment described in detail above.

Therefore, the invention is defined and delimited by the set of claims that follows.

1. Electrical impedance tomography equipment comprising a plurality of acquisition units (15, 27), as well as complementary elements and devices, the said acquisition units being provided with circuits for processing of low level analog signals picked up by a plurality of cables (13, 29) that interconnect the said equipment with the electrodes (14) applied to the patient, characterized in that the said acquisition units are provided substantially symmetrically in relation to at least one axis of symmetry (X, Y, Z).

2. Equipment, as claimed in claim 1, characterized in that the said at least one axis of symmetry is a vertical axis (Z).

3. Equipment, as claimed in claim 1, characterized in that the said complementary elements and devices are comprised in at least one plane (11, 25, 26) constituted by a printed circuit board, to which are electrically connected, in symmetrical fashion, the said acquisition units (15, 27).

4. Equipment, as claimed in claim 3, characterized in that the said acquisition units comprise two regions provided side by side, one of such regions comprising the circuits for analog processing of signals and the other comprising the circuits for digital processing of signals.

5. Equipment, as claimed in claim 3, characterized in that the said acquisition units (27) comprise a lower region (27a) containing the circuits for analog processing of signals and an upper region (27b) containing the circuits for digital processing of signals.

6. Equipment, as claimed in claim 4 or 5, characterized in that each acquisition unit (27) comprises at least one printed circuit board.

7. Equipment, as claimed in claim 6, characterized in that the said complementary elements and devices are comprised in a first plane (11, 25) and in a second plane (12, 26) provided parallel to one another.

8. Equipment, as claimed in claim 7, characterized in that the said first plane (11, 25) comprises devices that are substantially provided for processing of analog signals and the said second plane (11, 26) comprises devices that are substantially provided for processing of digital signals.

9. Equipment, as claimed in claim 1, characterized in that the said at least one axis of symmetry is a horizontal axis (X, Y).

10. Equipment, as claimed in claim 9, characterized by comprising two axes of symmetry (X, Y) defining a horizontal plane.

11. Equipment, as claimed in claim 1, characterized in that the said first (11, 25) and second (12, 26) planes are provided parallel in relation to the horizontal plane formed by the said two horizontal axes (X, Y).

12. Equipment, as claimed in claim 8, characterized in that the said first plane (11, 25) is electrically connected to the side (27c) adjacent to the region (27a) containing the said analog processing circuits, in the said acquisition units (27), and the said second plane (12, 26) is electrically connected to the side (27d) adjacent to the region (27b) containing the said digital processing circuits, in the said acquisition units.

13. Equipment, as claimed in claim 12, characterized in that the said acquisition units (15, 27) are provided between the first (25) and second (26) planes, and are symmetrically arranged in relation to at least one axis of symmetry (X, Y, Z).

14. Equipment, as claimed in claim 13, characterized in that the said acquisition units (15) are symmetrically arranged in relation to the vertical axis of symmetry (Z).

15. Equipment, as claimed in claim 13, characterized in that the said acquisition units (27) are evenly arranged along the borders of at least one of the said first (25) or second (26) planes.

16. Equipment, as claimed in claim 12, characterized in that the spatial arrangement of the boards that make up the said equipment constitutes a geometric solid.

17. Equipment, as claimed in claim 16, characterized in that the said first and second planes have the same dimensions and shape.

18. Equipment, as claimed in claim 17, characterized in that the said geometric solid is shaped as a prism whereof the sides are the acquisition units (15, 27) and which lower and upper bases are respectively the said first plane (11, 25) and the said second plane (12, 26).

19. Equipment, as claimed in claim 18, characterized in that the said geometric solid has the shape of a parallelepiped with a square base.

20. Equipment, as claimed in claim 12, characterized in that the said first and second planes have different dimensions.

21. Equipment, as claimed in claim 1, characterized in that the said first and second planes are interconnected by at least one interface board (32).

22. Equipment, as claimed in claim 21, characterized in that the said interface board (32) is positioned substantially at the center of the boards that constitute the said first (11, 25) and second (12, 26) planes.

23. Equipment, as claimed in claim 1, characterized in that the printed circuit board that constitutes the said first plane (25) comprises connecting elements (24) of the proximal ends of the said interconnection cables (29).

24. Equipment, as claimed in claim 23, characterized in that the said connecting elements (24) are arranged at a substantially central position on the said board (25).

25. Equipment, as claimed in claim 24, characterized in that the said connecting elements are connected to the acquisition units (27) by means of strips provided on the said board (25), the said strips (38a, 38b, 38c, 38d) being configured in order to have substantially identical lengths.
